(19) United States
(12) Patent Application Publication
(10) Pub. No.: US 2017/0095653 A1
(43) Pub. Date: Apr. 6, 2017

(54) UNIVERSAL MICROPORT

(71) Applicant: THE JOHNS HOPKINS UNIVERSITY, Baltimore, MD (US)

(72) Inventors: Juvenal Reyes, Cockeysville, MD (US); Michael Lim, Reisterstown, MD (US)

(21) Appl. No.: 15/284,716

(22) Filed: Oct. 4, 2016

Related U.S. Application Data
(60) Provisional application No. 62/237,646, filed on Oct. 6, 2015.

(51) Int. Cl. A61M 39/02; A61D 7/00; A61M 25/01

(52) U.S. Cl. CPC A61M 39/0208; A61M 25/0102; A61D 7/00; A61M 2039/0223

(57) ABSTRACT

The present invention is directed to a device that provides an implantable port, particularly for use with animal models, such as small animal models like mouse models. A microport device according to an embodiment of the present invention includes a reservoir and a catheter for delivery of substances held within the reservoir. The catheter can be barbed and can decrease in diameter from its proximal end to its distal end. The reservoir can include diaphragms for the delivery of substances to be held within the reservoir. The device can also include a sleeve that covers the device and a tip protector that covers the distal tip of the catheter.

### CROSS REFERENCE TO RELATED APPLICATION

This application claims the benefit of U.S. Provisional Patent Application No. 62/237,646 filed on Oct. 6, 2015, which is incorporated by reference, herein, in its entirety.

### FIELD OF THE INVENTION

The present invention relates generally to medical devices. More particularly, the present invention relates to a universal microport for intravenous, intra-arterial, and intra-ventricular access.

### BACKGROUND OF THE INVENTION

Intravenous, intra-arterial, and intra-ventricular access can be challenging. This challenge is increased when animal models, particularly small animal models, such as mouse models, require intravenous, intra-arterial, and intra-ventricular access.

Accordingly, there is a need in the art for a device to allow for intravenous, intra-arterial, and intra-ventricular access, especially in animal models.

### SUMMARY OF THE INVENTION

The foregoing needs are met, to a great extent, by the present invention which includes a device for delivery of a substance including a reservoir configured for holding an amount of a substance in an inner chamber defined by an outer wall of the reservoir. The device includes a catheter defining a lumen extending therethrough for delivering the substance, wherein the catheter defines outlets for dispensing the substance and includes anchors for holding the catheter in place. The device also includes a stylet configured to extend from a proximal end of the reservoir to a distal end of the catheter.

In accordance with an aspect of the present invention, the catheter further includes a decrease in diameter between a proximal end and the distal end. The anchors are placed randomly over a surface of the catheter. Alternately, the anchors are placed in a pattern over a surface of the catheter. The reservoir includes a small diaphragm and a large diaphragm. The device includes a means for moving the substance from the reservoir through the catheter and out of the outlets. A sleeve that envelopes the device can be used. A tip protector positioned at a distal end of the catheter can also be used. The device is configured for use in an animal model, a small animal model, and/or a mouse model.

In accordance with another aspect of the present invention, a device for delivery of a substance includes a reservoir configured for holding an amount of a substance in an inner chamber defined by an outer wall of the reservoir. The device includes a catheter defining a lumen extending therethrough for delivering the substance, wherein the catheter defines outlets for dispensing the substance. The device also includes anchors configured for holding the catheter in place.

In accordance with yet another aspect of the present invention, the catheter further includes a decrease in diameter between a proximal end and the distal end. The anchors are placed randomly over a surface of the catheter. The anchors are placed in a pattern over a surface of the catheter. The reservoir includes a small diaphragm and a large diaphragm. The device includes a means for moving the substance from the reservoir through the catheter and out of the outlets. The device can include a sleeve that envelopes the device. A tip protector is positioned at a distal end of the catheter. The anchors are positioned to impede movement of the device.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a side view of a microport according to an embodiment of the present invention.

FIG. 2 illustrates a side view of a microport including a sleeve and a tip protector, according to an embodiment of the present invention.

FIG. 3 illustrates a side, semi-sectional view of the microport of FIG. 1, according to an embodiment of the present invention.

FIG. 4 illustrates a side view of a mouse with the microport of the present invention implanted under its skin.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a device that provides an implantable port, particularly for use with animal models, such as small animal models like mouse models. A microport device according to an embodiment of the present invention includes a reservoir and a catheter for delivery of substances held within the reservoir. The catheter can be barbed and can decrease in diameter from its proximal end to its distal end. The reservoir can include diaphragms for the delivery of substances to be held within the reservoir. The device can also include a sleeve that covers the device and a tip protector that covers the distal tip of the catheter.

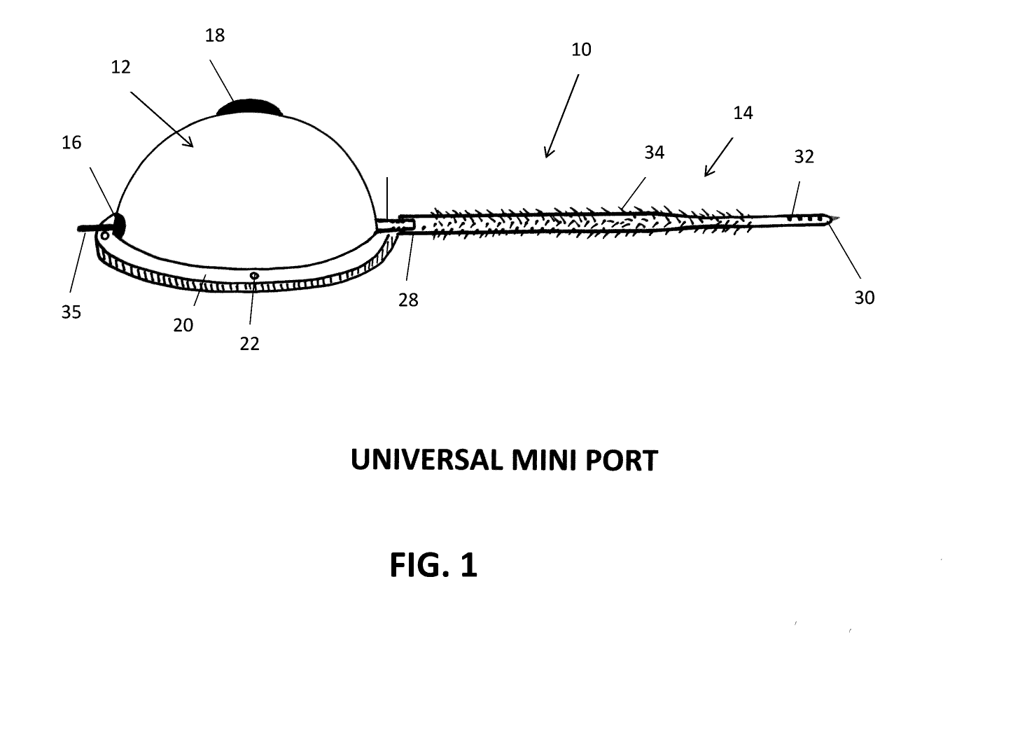

FIG. 1 illustrates a side view of a microport according to an embodiment of the present invention. The microport device 10 includes a reservoir 12 and a catheter 14. Small diaphragm 16 and large diaphragm 18 are used for creating flow for drug delivery, in a manner known to or conceivable by one of skill in the art. The diaphragms 16, 18 can be formed from rubber or a thermoplastic elastomer, or any other suitable material known to or conceivable by one of skill in the art. The diaphragms 16, 18 can also be used for delivery of substances to the reservoir 12 and for insertion and removal of the stylet, which will be described further herein. The reservoir 12 includes a base 20 that can be used to secure the microport in place via suture holes 22. The reservoir 12 can take a domed, cylindrical, rectangular, cubic, or any other suitable shape known to or conceivable by one of skill in the art. As illustrated herein, the reservoir takes a domed shape, but this is not meant to be considered limiting. The reservoir 12 includes an outlet port 24 that couples to catheter 14.

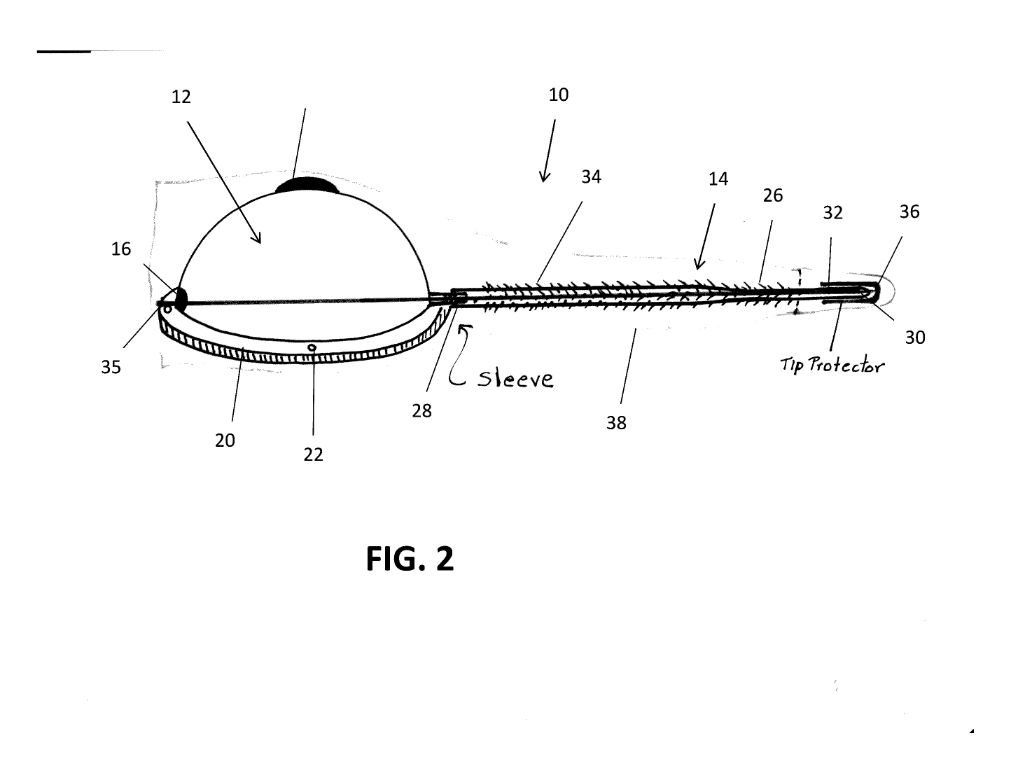

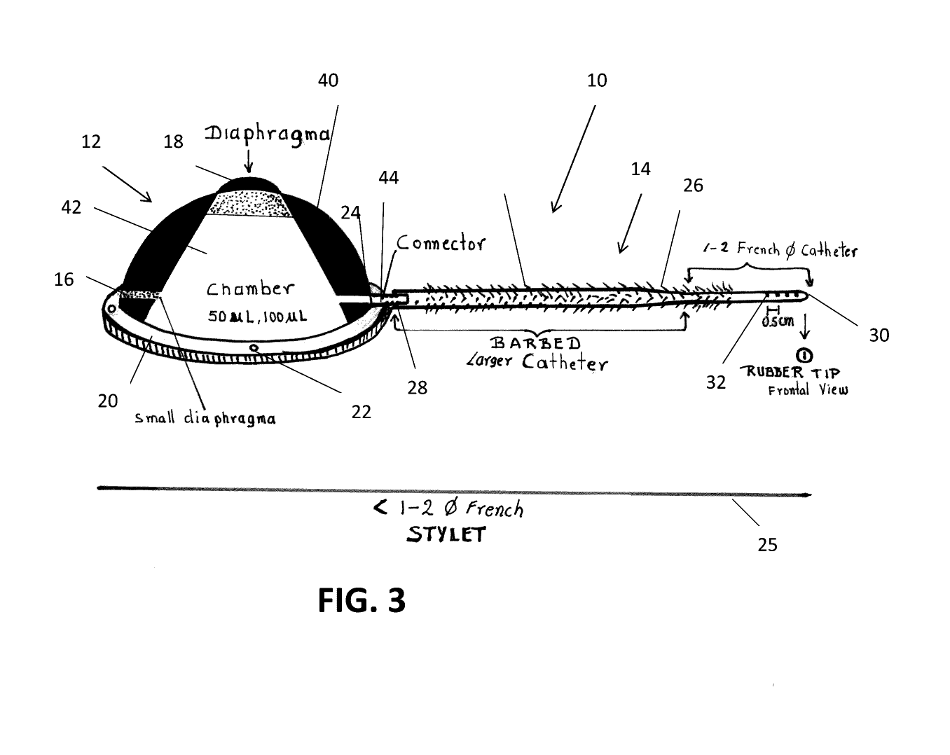

Further with respect to FIG. 1, the catheter 14 can include a tapered distal tip 26 which results in a decrease in diameter from the proximal end 28 of the catheter 14 to the distal end 30 of the catheter 14. The design of the distal end 30 of the catheter 14 minimizes backflow and the potential for obstruction. The catheter 14 also includes outlet ports 32 positioned just proximal to the distal end 30 of the catheter 14. The catheter defines a lumen extending therethrough that provides fluid communication between the reservoir and the outlets. The design of the distal end 30 of the catheter 14 minimizes backflow and the potential for obstruction. The outlet ports 32 can include a valve system so that fluid can exit but cannot backwash into the catheter 14. This minimizes the risk of blood clots forming within the catheter and eliminates the need for a heparin flush. Anchors 34 are positioned on the surface of the catheter 14. The anchors 34 serve to minimize risk of dislodgement of the catheter. The catheter 14 can be covered in anchors 34 or the anchors 34 can be positioned strategically on the surface of the catheter 14 in a pattern known to or conceivable by one of skill in the art. The reservoir 12 housing can be made from plastics, rubber, or any other suitable biocompatible material known to or conceivable by one of skill in the art. The catheter can be formed from a thermoplastic or any other suitable, biocompatible material known to or conceivable by one of skill in the art. The microport also includes stylet 35 which is only partially visible in FIG. 1 and will be described in further detail with respect to FIGS. 2 and 3.

FIG. 2 illustrates a side view of a microport including a sleeve and a tip protector, according to an embodiment of the present invention. As illustrated in FIG. 2, the device illustrated and described with respect to FIG. 1 can also include a tip protector 36 and a sleeve 38. The microport 10 includes stylet 35 that extends from the proximal end of the microport 10 to the distal end of the microport 10. The stylet 35 is used for insertion of the catheter 14 into the desired location. The stylet 35 is removed after the device in secured in place and the catheter 14 is in the desired location.

FIG. 3 illustrates a side, semi-sectional view of the microport of FIG. 1, according to an embodiment of the present invention. As illustrated in FIG. 3, the reservoir 12 can be designed to accommodate a certain amount of substance such as 50 μL or 100 μL. The reservoir 12 can be designed to hold an amount of substance in a range from 10 μL to 200 μL. A wall 40 of the reservoir 12 defines the inner chamber 42 that holds the substance. Diaphragms 16, 18 are also in communication with the inner chamber 42 in order to facilitate fluid flow out of the inner chamber and through the catheter 14. Connector 44 couples the reservoir 12 to the catheter 14. the catheter 12 can take any suitable size, however, it should be noted that the decreased distal end 30 of the catheter 14 is approximately a 1-2 French catheter size in certain embodiments. The catheter can also include outlet ports 32. If multiple outlet ports 32 are used approximately 0.5 cm of space is between each of the outlet ports and the next outlet port. As illustrated in FIG. 3, the catheter 14 includes four outlet holes. However, any suitable number of outlet holes known to or conceivable by one of skill in the art could also be used. It should also be noted that the valve system allows for fluid to flow out of the device but not back onto the device. This helps to prevent blood clots and to eliminate the need for heparin flush. Stylet 35 extends from the proximal end of the microport 10 to the distal end of the microport 10. The stylet 35 is used for insertion of the catheter 14 into the desired location. The stylet 35 is removed after the device is secured in place and the catheter 14 is in the desired location. The stylet 35 can take the form of any suitable stylet 35 known to or conceivable by one of skill in the art. In some embodiments, the stylet 35 can take the form of a 1-2 French stylet. The stylet 35 can be formed from metal, plastic, wire, or any other suitable material known to or conceivable by one of skill in the art.

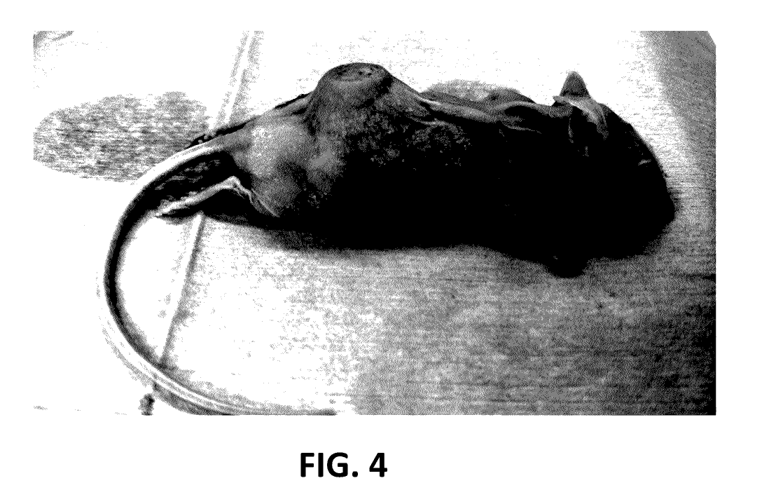

FIG. 4 illustrates a side view of a mouse with the microport of the present invention implanted under its skin. The reservoir can be seen under the skin of the mouse as can the catheter extending from the port.

It should be noted that the microport according to the present invention can be used for a number of different applications, such as intravenous, intraventricular, and intraarterial access. The device can be used in animal models, particularly small animal models, such as mice and rats. However, this is not meant to be considered limiting and the device could also be used in larger animals and humans.

The many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A device for delivery of a substance comprising:
a reservoir configured for holding an amount of a substance in an inner chamber defined by an outer wall of the reservoir;
a catheter defining a lumen extending therethrough for delivering the substance, wherein the catheter defines outlets for dispensing the substance and comprises anchors for holding the catheter in place; and
a stylet configured to extend from a proximal end of the reservoir to a distal end of the catheter.

2. The device of claim 1 wherein the catheter further comprises a decrease in diameter between a proximal end and the distal end.

3. The device of claim 1 wherein the anchors are placed randomly over a surface of the catheter.

4. The device of claim 1 wherein the anchors are placed in a pattern over a surface of the catheter.

5. The device of claim 1 wherein the reservoir includes a small diaphragm and a large diaphragm.

6. The device of claim 1 further comprising a means for moving the substance from the reservoir through the catheter and out of the outlets.

7. The device of claim 1 comprising a sleeve that envelopes the device.

8. The device of claim 1 comprising a tip protector positioned at a distal end of the catheter.

9. The device of claim 1 comprising the device being configured for use in an animal model.

10. The device of claim 1 comprising the device being configured for use in a small animal model.

11. The device of claim 1 comprising the device being configured for use in a mouse.

12. A device for delivery of a substance comprising:
a reservoir configured for holding an amount of a substance in an inner chamber defined by an outer wall of the reservoir;
a catheter defining a lumen extending therethrough for delivering the substance, wherein the catheter defines outlets for dispensing the substance; and
anchors configured for holding the catheter in place.

13. The device of claim 12 wherein the catheter further comprises a decrease in diameter between a proximal end and the distal end.

14. The device of claim 12 wherein the anchors are placed randomly over a surface of the catheter.

15. The device of claim 12 wherein the anchors are placed in a pattern over a surface of the catheter.

16. The device of claim 12 wherein the reservoir includes a small diaphragm and a large diaphragm.

17. The device of claim 12 further comprising a means for moving the substance from the reservoir through the catheter and out of the outlets.

18. The device of claim 12 comprising a sleeve that envelopes the device.

19. The device of claim 12 comprising a tip protector positioned at a distal end of the catheter.

20. The device of claim 12 wherein the anchors are positioned to impede movement of the device.
